(19) United States
(12) Patent Application Publication
(10) Pub. No.: US 2006/0009731 A1
(43) Pub. Date: Jan. 12, 2006

(54) Electric energy auxiliary device of transdermal drug delivery patch

(76) Inventors: Tsai-Fu Wu, Min-Hsiung (TW); Sheng-Yu Tseng, Min-Hsiung (TW); Wen-Hao Chang, Min-Hsiung (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/012,212

(22) Filed: Dec. 16, 2004

(30) Foreign Application Priority Data
Jul. 9, 2004 (TW) 93120566

(51) Int. Cl. A61N 1/30

(52) U.S. Cl. 604/20

(57) ABSTRACT

An electric energy auxiliary device of transdermal drug delivery patch is provided which includes an upper electrode, a lower electrode, and a drug storage area between two electrodes, generating a kinetic energy in the pharmaceutical compound in the drug storage area toward the skin by a electric field between two electrodes. For the pharmaceutical compound with the same polarity, the pharmaceutical compound is pushed by the electric energy interacted with polar the pharmaceutical compound so as to deliver the drug the human body. The lower electrode generates an ion channel with electroporation on the surface of skin. By using the change of electric field on the surface of skin, a temporary ion channel is generated in the cells where the pharmaceutical compound enters the systemic circulation of human body via skin. This temporary ion channel allows large compound to pass into the skin of human body.

### BACKGROUND OF THE INVENTION

1. Field of the Invention

The invention relates to a drug delivery technology, and, more particularly, to an electric energy auxiliary device of transdermal drug delivery patch which can be applied to the treatment for systemic disease or the field of transporting the pharmaceutical compounds or ions by the transdermal way.

2. Description of the Prior Art

Recently, transdermal route has vied with oral and injection treatments as the most successful innovative research region in drug delivery. It will be a future choice for systemic delivery of protein and peptide with large compound, which usually can only be delivered by parenteral therapy. In worldwide transdermal patch market, there exist only ten drugs, such as scopolamine, nitroglycerine, clonidine, etc. In these drugs, their delivery is generally passive, which has a low skin-penetration capability. Thus, electrically assisted methods are required to improve drug delivery efficiency of the passive diffusion.

In general, drug delivery involves either chemical or physical processing, or both, including the oral treatment, intravenous, intramuscular, subcutaneous, transdermal patch, iontophoresis, electroporation. With chemical processing, drug may be remained in skin surface or interacted with human body, resulting in hurting organism. Compared with chemical processing, the physical one is more safe for drug delivery, and it is usually in a transdermal way. Transdermal drug delivery (TDD) is divided into two categories: active and passive ones. To attain safe and comfortable drug delivery, active methods are usually used to deliver drugs with electrical assistance. In conventional electrical assistance, processes of iontophoresis and electroporation are used for TDD. The iontophoretic delivery system is shown in FIG. 1, which comprises a set of positive electrode 10 and negative electrode 12 positioned on the surface of skin 14. By using the attraction and the pushing between the electric field and the pharmaceutical compounds 16, the pharmaceutical compounds 16 are delivered into the blood vessel 20 of the deeper stratum 18 (systemic circulation of human body). Another iontophoretic delivery system comprises their electrodes having two sets of metallic film for applying pulsed voltages to the patch to increase drug-penetration capability. Skin electroporation creates transient aqueous pore in the lipid bilayers by application of short electrical pulses processes of electroporation can be illustrated by FIGS. 2(a) to 2(e). The normal cell membrane 70 is shown in FIG. 2(a). The cell membrane 70 is excited by short electrical pulses resulting in irregular compound structure shown in FIG. 2(b). If the water-solube compound concentration outside the cell membrane 70 is high, the outer of adipose stratum will be destroyed causing the cell membrane 70 notched, shown in FIG. 2(c). The cell membrane 70 with a temporary hydrophobic pore 84 is formed shown in FIG. 2(d). The cell membrane 70 with a hydrophilic pore 86 is formed after membrane restructuring, shown in FIG. 2(e). These pores provide pathways for penetration, from which the charged compounds and ions travel straightforwardly through the cell membrane 70. After the voltage of electroporation is removed, the cell membrane 70 is restored to be a normal cell membrane of FIG. 2(a).

Electrically-activates devices have been described in U.S. Pat. No. 6,377,848 shown in FIG. 3, comprising a control circuitry, a set of electrodes and the drug. The devices transport drug into the human baby by an iontophoretic drug delivery. The devices include a power source 28, a patch 30 and the electronic interconnectors 32 of the power source 28. The patch 30 includes the positive electrode 34 and the negative electrode 36. The patch 30 includes the positive electrode 34 and the negative electrode 36 positioned respectively in the reservoirs 38 and 40. The patch 30 is attached to the surface of the skin 42. The power source 28 provides the electric energy to the electrodes 34 and 36 by the electronic interconnectors 32. The polarity of the electrode 36 is negative, and the polarity of the electrode 34 is positive. Since the drug in the reservoirs 38 and 40 are the ionized negatively drug, the negative polarity of the electrode 36 is repulsed with the negative polarity of the ionized drug due to the same charge, thereby generating a force across the skin 42. The drug is delivered into the deeper tissues of the skin 42 by this force. When the pharmaceutical compound is delivered into the deeper tissues of the skin 42, the negative polarity of the ionized drug is neutralized due to electrolysis stratum in the most bottom of stratum corneum. The pharmaceutical compound across stratum corneum of the skin is unable to penetrate more deeper, only maintaining in the corneum. To solve this problem, an iontophoretic delivery system is provided. The positive ions in the electrolysis stratum is interacted with the negative electrode, thereby attracting the positive ions from the electrolysis stratum, so as the negatively ionized drug can penetrate through skin and enter the blood vessel.

However, a problem may sexist because it takes some time to reach the treatment concentration in the blood vessel when using the patch with an iontophoretic delivery method. In additional, this two-electrodes structure may cause the polarization of electrode. When the overpotential is generated, it is uncomfortable for the patient. As these problems previously, the poisonous material will be generated to cause the injury for the patient.

### SUMMARY OF THE INVENTION

An object of the present invention is to provide an electric energy auxiliary device of transdermal drug delivery patch with a multi-electrode structure having drug delivery efficiency of iontophoresis, electroporation, and natural diffusion.

Another object of the present invention is to provide an electric energy auxiliary device of transdermal drug delivery patch that provides a strongly thrust by using two-electrodes structure to deliver the pharmaceutical compounds into the human body readily.

Another object of the present invention is to provide an electric energy auxiliary device of transdermal drug delivery patch, thereby generating the rotational electric field by the lower electrode, preventing the skin from the single direction of electric field so as to reduce the polarization of electrodes.

Another object of the present invention is to provide an electric energy auxiliary device of transdermal drug delivery patch, not being subjected to the size and hydrophile of pharmaceutical compound so as to improve effectiveness of drug delivery.

Another object of the present invention is to provide an electric energy auxiliary device of transdermal drug delivery patch, which is used in drug delivery system with the polar and neutral pharmaceutical compounds.

On one aspect of the present invention, an electric energy auxiliary device of transdermal drug delivery patch is provided which includes an upper electrode, a lower electrode, and a drug storage area between two electrodes, generating a kinetic energy in the pharmaceutical compounds in the drug storage area toward the skin by an electric field between two electrodes. For the pharmaceutical compounds with the same polarity, the pharmaceutical compounds are pushed by the electric energy interacted with the polar pharmaceutical compound so as to deliver the drug into the human body. The lower electrode generates an ion channel with electroporation on the surface of skin. By using the change of electric field on the surface of skin, a temporary ion channel is generated in the cells where the pharmaceutical compounds enter the systemic circulation of human body via skin. This temporary ion channel allows the large compound to pass into the skin of human body.

In another aspect of the present invention, a thin film is provided to control the release rate of the pharmaceutical compounds. The thin film is attachable to the adhesion layer of skin, and covered over the backing of the outer surface of the upper electrode.

### BRIEF DESCRIPTION OF THE DRAWINGS

These and other objects, features and advantages of the present invention can best be understood by reference to the detailed description of the preferred embodiments set forth below taken with the drawings, in which:

FIG. 1 is a schematic view of the iontophoretic drug delivery system in accordance with the prior art;

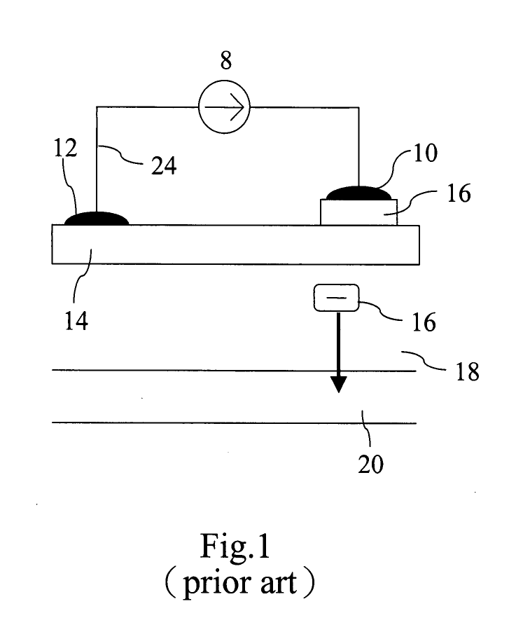

FIGS. 2(a) through 2(e) are schematic views of a cell membrane excited by short electrical pulses;

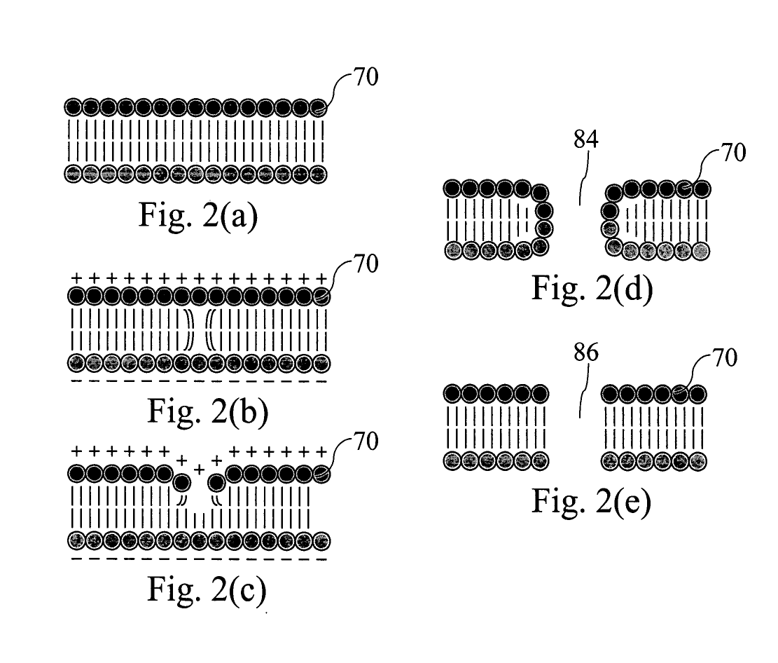

FIG. 3 shows a block diagram of an iontophoretic drug delivery device in accordance with the U.S. Pat. No. 6,377,848;

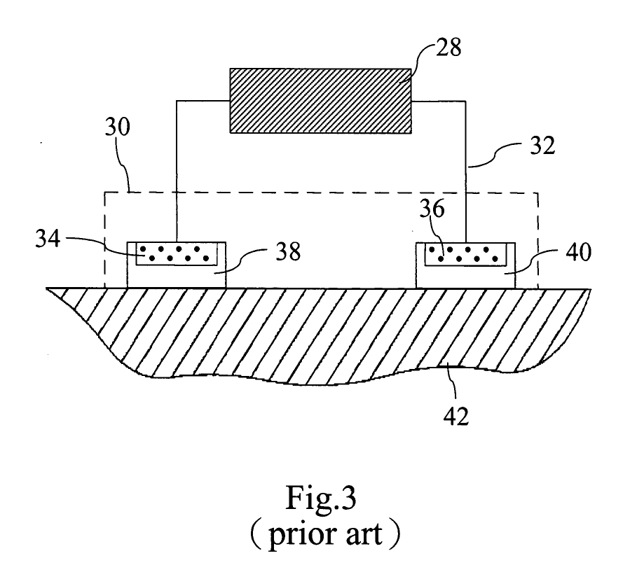

FIG. 4 shows a block diagram of electric energy auxiliary device of transdermal drug delivery patch in accordance with the present invention;

FIG. 5 is a moving diagram illustrating the charged particles in uniform electric field in accordance with the present invention;

FIG. 6 is a motion diagram illustrating the charged particles in uniform magnetic field in accordance with the present invention;

FIG. 7 is a diagram illustrating the positive particles forced by an electric field in accordance with the present invention;

FIG. 8 a graph of the magnetic flux \vec{B} versus the electric field in the time-varied electric field in accordance with the present invention;

FIG. 9 a vector diagram of the positive particles versus the magnetic field in the time-varied electric field in accordance with the present invention;

FIG. 10 a vector diagram of the force \vec{F}_{c} combined the force of electric field \vec{F}ₑ with the force of magnetic field \vec{F}ₘ in accordance with the present invention;

FIG. 11 a vector diagram of two active electrodes versus the force of an ion in accordance with the present invention;

FIG. 12 shows a block diagram of two active electrodes in accordance with the present invention;

FIG. 13 shows a block diagram illustrating a single set of upper electrode and two sets of lower electrodes in accordance with the present invention;

FIG. 14 is a drive timing diagram illustrating the positive electrode drug transport in accordance with the present invention;

FIG. 15 is a relative position diagram of electric field component having xy plane formed by electric field versus a pharmaceutical compound in accordance with the present invention;

FIG. 16 is a relative position diagram of electric field component having xz plane formed by electric field versus a pharmaceutical compound in accordance with the present invention;

FIG. 17 is a relative position diagram of electric field component having xy, yz, and zx plane formed by electric field versus a pharmaceutical compound in accordance with the present invention;

FIG. 18 is a vector diagram of the resulting force \vec{F}_{c1} interacting with \vec{F}ₑ₁ and \vec{F}ₘ₁ in accordance with the present invention;

FIG. 19 is a vector diagram of the resulting force \vec{F}_{c2} interacting with \vec{F}ₑ₂ and \vec{F}ₘ₂ in accordance with the present invention;

FIG. 20 is a vector diagram of the resulting force \vec{F}_{c3} interacting with \vec{F}_{E} and \vec{F}ₘₓ, \vec{F}_{my} and \vec{F}_{mz} in accordance with the present invention;

FIG. 21 shows a block diagram illustrating a single set of upper electrode and three sets of lower electrodes in accordance with the present invention;

FIG. 22 shows a block diagram illustrating a single set of upper electrode and four sets of lower electrodes in accordance with the present invention;

FIG. 23 is a diagram of the driving way illustrating three sets of lower electrodes in accordance with the present invention;

FIG. 24 is a diagram of the driving way illustrating four sets of lower electrodes in accordance with the present invention;

FIG. 25 is a drive timing diagram illustrating the negative electrode drug transport in accordance with the present invention;

FIG. 26 is a relative position diagram of electric field component having xy plane formed by electric field versus a pharmaceutical compound in accordance with the present invention;

FIG. 27 is a relative position diagram of electric field component having xz plane formed by electric field versus a pharmaceutical compound in accordance with the present invention;

FIG. 28 is a relative position diagram of electric field component having xy, yz, and zx plane formed by electric field versus a pharmaceutical compound in accordance with the present invention;

FIG. 29 a vector diagram of the resulting force \vec{F}_{c4} interacting with \vec{F}ₑ₄ and \vec{F}ₘ₄ in accordance with the present invention;

FIG. 30 a vector diagram of the resulting force \vec{F}_{c5} interacting with \vec{F}ₑ₅ and \vec{F}ₘ₅ in accordance with the present invention; and

FIG. 31 is a vector diagram of the resulting force \vec{F}_{c6} interacting with \vec{F}_{E} and \vec{F}ₘₓ, \vec{F}_{my} and \vec{F}_{mz} in accordance with the present invention.

### DESCRIPTION OF THE PREFERRED EMBODIMENTS

The present invention provides an electric energy auxiliary device of transdermal drug delivery patch with a multi-electrode structure, which improves the active and passive Transdermal drug delivery (TDD) of the prior art. When using the patch of the present invention, the present invention provides a strongly thrust by using two-electrode structure to deliver the pharmaceutical compound into the human body readily, thereby preventing the polarization of electrodes from the treatment, so as to reduce uncomfortable feeling for the patient.

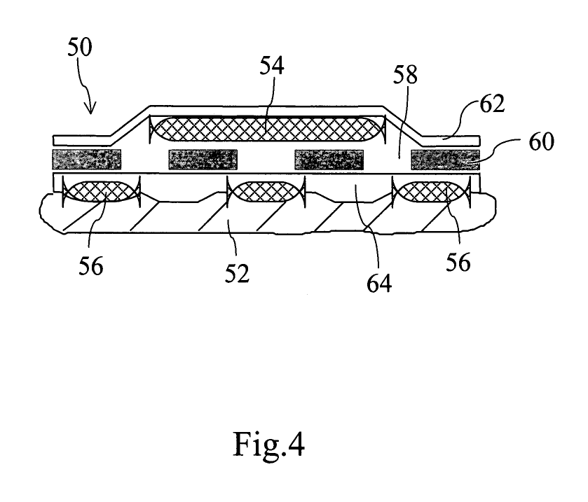

An electric energy auxiliary device 50 of transdermal drug delivery patch attached to the skin 52 of human body in accordance with the present invention, as shown in FIG. 4, includes a two-electrode structure, the upper electrode 54 and the lower electrode 56, which are respectively formed of any shape of electrode array, The time-varied voltage is applied on the upper electrode 54 and the lower electrode 56. A drug storage area 58 is placed between the upper electrode 54 and the lower electrode 56. A thin film 60 is positioned between the drug storage area 58 and the lower electrode 56 to control the release rate of pharmaceutical compound in the drug storage area 58. A layer of a backing 62 is covered over the outer surface of the upper electrode 54. An adhesion layer 64 is positioned between the lower electrode 56 and the thin film 60 to cover the top surface of the lower electrode 56 so as to attach to the skin of human body. In the electric energy auxiliary device 50, by using the rotational electric field by the upper electrode 54 and the lower electrode 56, a kinetic energy is generated in pharmaceutical compound in the drug storage area 58 toward the skin 52. The lower electrode 56 generates a temporary ion channel with electroporation on the surface of skin 52, allowing pharmaceutical compound across through the skin 52 easily, so as to deliver the drug into the human body rapidly.

The present invention has been already disclosed. The detailed principle of the present invention is described herein. The present invention can be presented to a user, and the user can visually understand the present invention.

In nature, the electromagnetic force, the gravitational force, the strong force, and the weak force are called the fundamental forces. The electromagnetic force and daily life are closely related. Also, the electromagnetic force is the power source of Transdermal Drug Deliver. The action force relationship between the electromagnetic force and the matter relates to the amount of charge carried by the matter. When the charged particle is positioned in the uniform electromagnetic field, the force is represented by the following equation (1),
\vec{F}=q(\vec{E}+\vec{V}×\vec{B}) (1)

where \vec{F} is the force which the charged particle is subjected to the uniform electromagnetic field, which combines the electric filed force with the magnetic field force, q is the charge amount of charged particle, \vec{E} is the electric field intensity, \vec{V} is the moving velocity after the charged particle is subjected to the electric filed, and \vec{B} is magnetic flux density. The forces between the electric filed, magnetic field, and the matter are described as the followings:

(a) The force which the charged particle is subjected to the electric filed

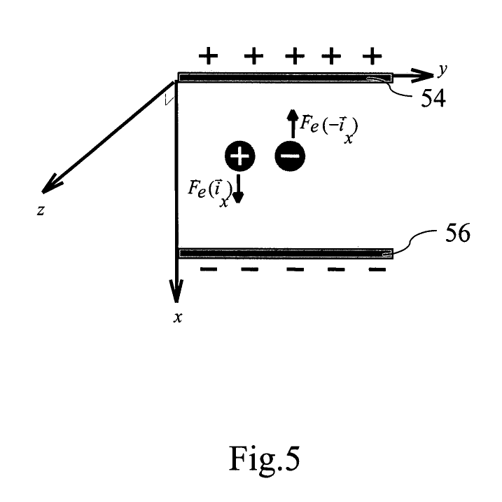

The movement which the charged particle is subjected to the uniform electric field is shown in FIG. 5. \vec{F}ₑ is the force which the electric field is applied on the charged particle. X, Y and z are the directions of right angle coordinate. When the charged particle is positioned in the uniform electric field, the positive charged particle is moving toward the negative electrode (lower electrode) 56 due to the electric field, and the negative charged particle is moving toward the positive electrode (upper electrode) 54. The charged particle in the uniform electric field is subjected to a force. The force is determined by the following equation (2).
\vec{F}ₑ=q\vec{E} (2)

Where \vec{F}ₑ is the force which the charged electric field is applied on the charged particle, and \vec{E} is the electric field intensity.

(b) The force which the charged particle is subjected to the magnetic field

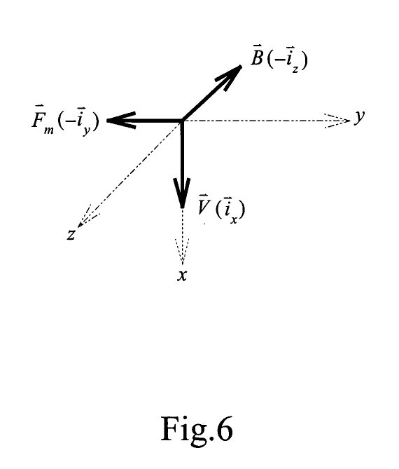

The movement which the charged particle is subjected to the uniform magnetic field is shown in FIG. 6. \vec{F}ₑ is the force which the electric field is applied on the charged particle. X, Y and z are the directions of right angle coordinate. When the charged particle is positioned in the uniform magnetic field and the charged particle is subjected to the magnetic effect, the force, which is perpendicular to the magnetic field and the moving direction, is produced. The force is determined by the following equation (3).
\vec{F}ₘ=q\vec{V}×\vec{B} (3)
where \vec{F}ₘ is the force which the magnetic field is applied on the charged particle, q is the charge amount of charged particle, \vec{V} is the moving velocity of the charged particle, and \vec{B} is magnetic flux density. According to the aforementioned equation, it found that the forces of electric field and the force of magnetic field are produced in sequence when a charged particle is positioned in the uniform electromagnetic field. The total force, which the charged particle is subjected to the electromagnetic field, is obtained by combining with these two forces. The total force is determined by the following equation (4).
\vec{F}=\vec{F}ₑ+\vec{F}ₘ=q(\vec{E}+\vec{V}×\vec{B}) (4)

If a positive particle is positioned in the electric field changing as the time, the relationship between the time-varied electric field and the magnetic field is determined by the Maxwell equation, as the following equation (5),

$$∇ \times \vec{E} = - \frac{\partial \vec{B}}{\partial t} \qquad ( 5 )$$

where \vec{E} is the electric field changing as the time, and \vec{B} is magnetic flux density. According to the equation (5), a time-varied electric field in the space can induce the magnetic field changed by the time, and the resulting force is obtained by combining the force of the electric field with the electromagnetic field.

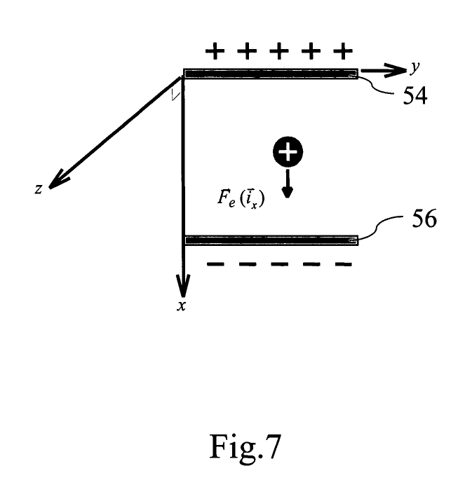

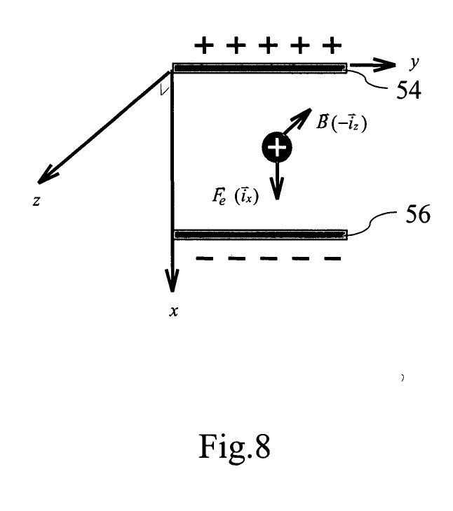

According to the Maxwell equation, if a charged particle is positioned in the time-varied electric field, the charged particle is subjected to the electric field first, and the force \vec{F}ₑ is produced, as shown in FIG. 7. The force of electric field is profiled on the xy plane. The vector \vec{E}ᵤ of electric field is represented by the equation (6).
\vec{E}ᵤ=\vec{E}ₓ(x,y,t)\vec{i}ₓ+E_{y}(x,y,t)\vec{i}_{y} (6)
where \vec{E}ᵤ is the vector of electric field, Ey is the vector component of axis y in the electric field \vec{E}ᵤ, and Ex is the vector component of axis x in the electric field \vec{E}ᵤ. Since \vec{E}ᵤ is the time-varied electric field, the equation (6) is conducted into the equation (5). The math relationship equation between the time-varied electric field and the time-varied magnetic field is represented by the equation (7).

$$( \frac{\partial {E}_{y}}{\partial x} - \frac{\partial {E}_{x}}{\partial y} ) {\vec{i}}_{z} = - \frac{ⅆ}{ⅆ t} \vec{B} {\vec{i}}_{z} \qquad ( 7 )$$

where ∂E_{y}/∂x is the partial derivative of variable of x in E_{y} function, ∂Eₓ/∂y is the partial derivative of variable of y in Eₓ function, and \vec{i}_{z} is the vector of unit direction of axis z. The induced magnetic flux \vec{B} and the vector of electric field are represented in FIG. 8.

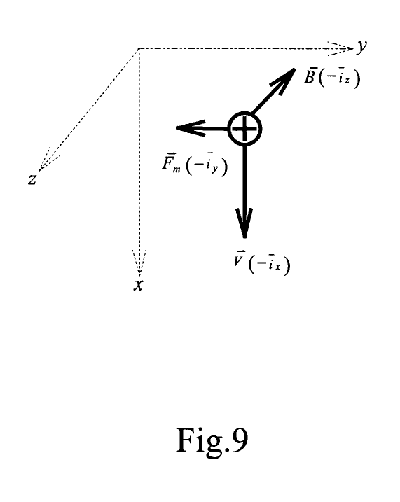

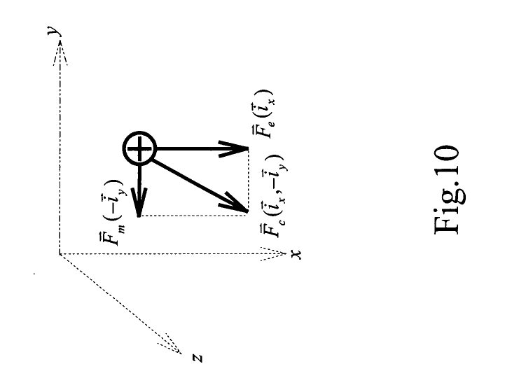

According to the equation (7), when the time-varied electric field generates an alternating magnetic filed \vec{B} (−\vec{i} z) in the space, there is a force \vec{F}ₘ applied on the charged particle. The vector relationship between the force \vec{F}ₘ and the magnetic flux is shown in FIG. 9. \vec{v} (\vec{i}ₓ)is the moving velocity of displacement produced by the charged particle in the electric field. The moving charged particle is subjected to the magnetic force \vec{F}ₘ in the magnetic field. According to the equation (3), \vec{F}ₘ of vector of direction is the direction of negative axis y. According to the aforementioned analysis, it found that a charged particle positioned in the alternating electric field is subjected to the force of electric field and the induced force of magnetic field simultaneously. The magnitude and direction of resulting force are obtained by the equation (1). Their vector relationship is shown in FIG. 10.

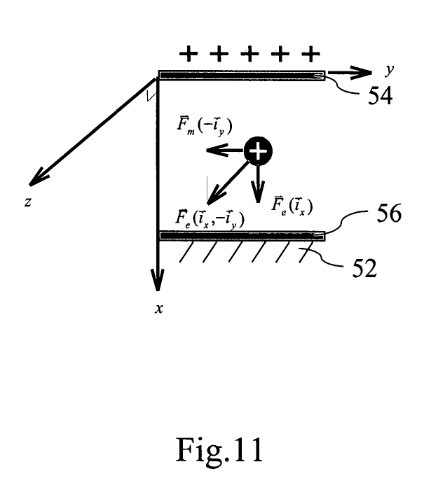

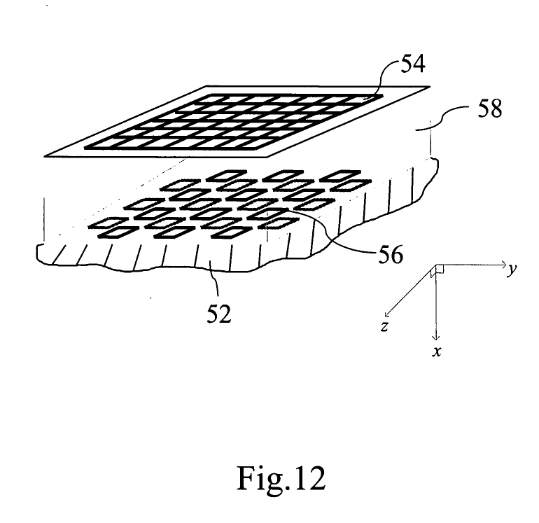

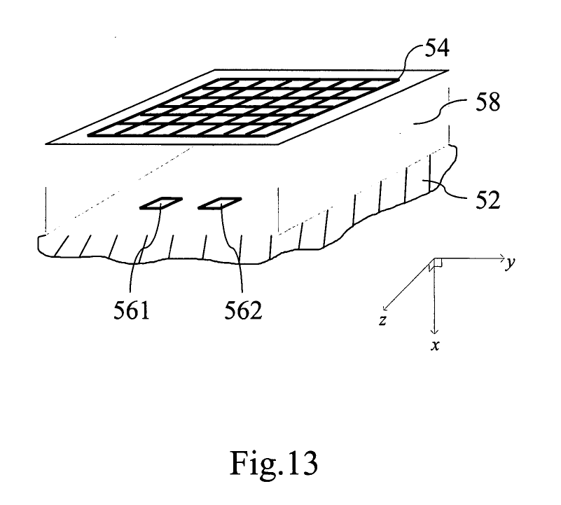

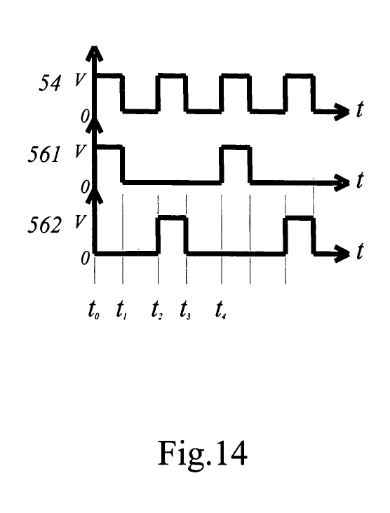

To sum up, the upper electrode 54 and the lower electrode 56 of the present invention are respectively positioned on the xy planes, and the pharmaceutical compound 16 with the polarity is positioned between two electrodes 54 and 56. The time-varied voltage is applied on the upper electrode 54 and the lower electrode 56. The space, which is the drug positioned in, exerts the force of electric field and the force of magnetic field simultaneously. The resulting vector of two forces is profiled on the xy plane. Therefore, the patch is attachable to the skin of human 52. The relative position of skin of human 52 on coordinate axis is on the xy plane, and is adjacent with the lower electrode 56 in order to move the pharmaceutical compound toward the skin 52 by the resulting electromagnetic force. This resulting force makes the pharmaceutical compound moving toward the surface of skin, shown in FIG. 11. In the present invention, the structure of lower electrode is the combination of multi-electrode. This set of multi-electrode is contacted with the skin directly. When an external voltage is applied on this set of electrodes, a temporary ion channel is produced in the obstacle cell of surface of skin. The pharmaceutical compound can pass through this channel into the human body for delivering the drug including the larger or small compound. The upper electrode 54 is called the electromagnetic electrode, and the lower electrode 56 is called the electroporation electrode. The architecture is shown in FIG. 12. In order to simplify the moving principle of the present invention, only one set of electrode is positioned in the upper electrode, and two sets of electrodes are positioned in the lower electrode. This combination is described by the architecture of electrodes of FIG. 13 and the drive timing of FIG. 14

In order to describe the moving principle of the present invention, a single set of upper electrode (the electromagnetic electrode) 54 and two sets of lower electrode (electroporation electrodes) are detailed shown in FIG. 13.

(I) The Drug with Positive Polarity

When the polarity of pharmaceutical compound which is going to deliver the human body is positive, the time-varied electromagnetic field is produced if the drug is positioned between the upper electrode and the lower electrode and the time-varied voltage is applied on these two electrodes, so that the pharmaceutical compound has the kinetic energy moving toward the skin. For the lower electrode, due to the rotational electric field applied by the alternating voltage, an ion channel is formed in the surface of skin, so that the pharmaceutical compound is delivered into the human body more easily. For the combination set of the alternating electric field and the rotational alternating electric field on the patch, the various combinations are formed, depending on the number of upper electrode and lower electrode. In order to simplify the moving principle of the present invention, only one set of electrode is positioned in the upper electrode, and two sets of electrodes are positioned in the lower electrode. This combination is described by the architecture of electrodes of FIG. 13 and the drive timing of FIG. 14. As shown in FIG. 14, each drive period has four states. There are the differences of two states t₀˜t₁ and t₂˜t₃ in four states. There is no voltage applied on the lower electrodes 561 and 562. The polarities of other electrodes are not changed. The drive timings t₀˜t₁ and t₂˜t₃ are moving the same way. The lower electrode 561 in the electric field is changed into the lower electrode 562, depending on the different state. According to the aforementioned, the movement ways of t₀˜t₂ and t₂˜t₄ are moving similar. In the following, the movement ways of t₀˜t₂ are only described.

(I) State 1 (t=t₀˜t₁)

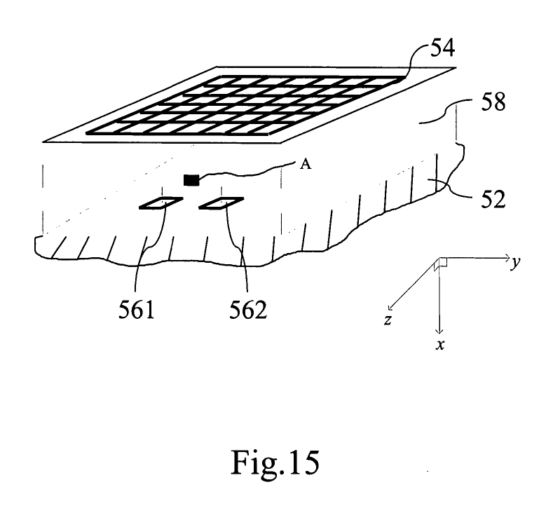

In this state, for the upper electrode 54 and the lower electrode 561, the positive voltages are applied on their voltage levels, and the voltage level and the timing-varied are the same. The variations in the electric field and the magnetic field are not changed between these two electrodes. For the lower electrode 562, the applied voltage is zero level of voltage. There is a variation of voltage between the lower electrode 562 and the upper electrode 54. The variation of voltage is changed between these two electrodes, depending on the plane position of the electrodes 561 and 562. The plane position of pharmaceutical compound on the lower electrode 561 and 562 is changed as three different areas, respectively, as shown in FIGS. 15, 16 and 17. In these figures, it can be found that, the vector of electric field is represented by the right-angled coordinate axis. The three different areas having the electric field profiles are analyzed as followings:

(a) The force which is applied on the pharmaceutical compound by the component of electric field having xy coordinate plane, as shown in FIG. 15, when a pharmaceutical compound A is positioned on the xy coordinate plane of lower electrode 561 and 562, a component of electric field is produced on this coordinate plane, and this component of electric field is profiled on the xy coordinate plane. The component of electric field caused by the lower electrode 562 to the upper electrode 54 is represented by the following equation (8).
\vec{E}_{UB}=\vec{E}_{xB}(x,y,t)\vec{i}ₓ+E_{yB}(x,y,t)\vec{i}_{y} (8)
where \vec{E}_{UB} is the vector of electric field profile caused by the upper electrode 54 to the lower electrode 562, E_{xB} is the component of \vec{E}_{UB} on x coordinate axis, E_{yB} is the component of \vec{E}_{UB} on y coordinate axis, and \vec{i}ₓ and \vec{i}_{y} are the unit vectors of the right-angled coordinate axis.

After the relationship of electric field vector is obtained, the equation (8) is calculated according to the equation (5). The following relationship equation is obtained.

$$( \frac{\partial {E}_{yB}}{\partial x} - \frac{\partial {E}_{xB}}{\partial y} ) {\vec{i}}_{z} = - \frac{ⅆ}{ⅆ t} B {\vec{i}}_{z} \qquad ( 9 )$$

where ∂E_{yB}/∂x is taking x as the partial derivative of component of \vec{E}_{UB} on y axis, ∂E_{xB}/∂y is taking y as the partial derivative of component of \vec{E}_{UB} on x axis, d/dt B is taking the differential of time by the magnetic flux when the electric field is changed.

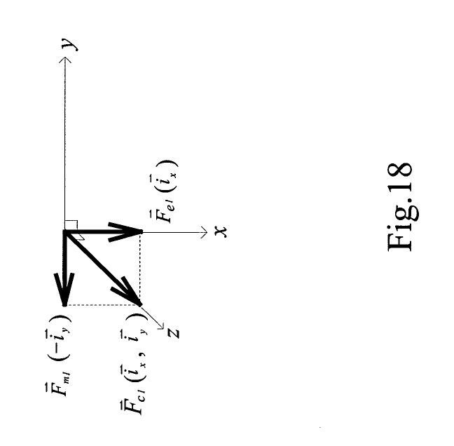

According to the equation (9), a magnetic field \vec{B} is induced by the time-varied electric field. When the pharmaceutical compound is positioned in the electric filed and the magnetic field inducted by the electric field, the pharmaceutical compound produces a force \vec{F}ₑ₁ of electric field by the electric field of upper electrode 54, thereby making the pharmaceutical compound downwardly moving with a velocity v. When this force \vec{F}ₑ₁ affects the moving pharmaceutical compound, the pharmaceutical compound is subjected to a force \vec{F}ₘ₁ in the magnetic field induced by the electric field. The magnitude and direction of this force \vec{F}ₘ₁ of magnetic field are determined by the equation (3). According to the above-mentioned analysis, it found that the pharmaceutical compound is subjected to the force \vec{F}ₑ₁ of electric field and the force \vec{F}ₘ₁ of magnetic field, represented by the equation (4), a force \vec{F}_{c1} is interacted with these two forces. The magnitude and direction of this force \vec{F}ₑ₁ are the magnitude and direction of the vectors of \vec{F}ₑ₁ and \vec{F}ₘ₁ plus each other, as shown in FIG. 18. In FIG. 18, it is seen that the direction of force \vec{F}_{c1} is pointed toward the directions of lower electrode 561 and 562. If the bottom of lower electrode 561 and 562 is positioned on the skin, a force \vec{F}_{c1} is produced. According to the above-mentioned analysis, a force is produced by the electric field profiled on the xy coordinate plane, thereby having the pharmaceutical compound with the kinetic energy moving towards the skin.

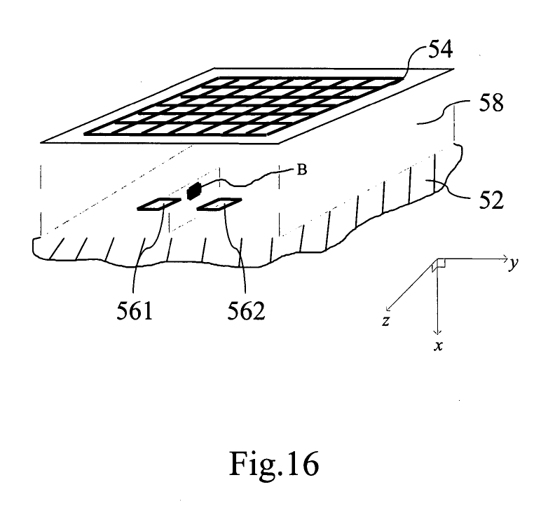

(b) The force which is applied on the pharmaceutical compound by the component of electric field having xz coordinate plane, as shown in FIG. 16, when a pharmaceutical compound B is positioned on the xz coordinate plane of lower electrode 561 and 562, a component of electric field is produced on this coordinate plane, and this component of electric field is profiled on the xz coordinate plane. The component of electric field caused by the lower electrode 562 from the upper electrode 54 is represented by the following equation (10).
\vec{E}_{UB}=E_{xB}(x,y,t)\vec{i}ₓ+E_{zB}(x,y,t)\vec{i}_{z} (10)
where \vec{E}_{UB} is the vector of electric field profile caused by the upper electrode 54 to the lower electrode 562, E_{xB} is the component of \vec{E}_{UB} on x coordinate axis, E_{yB} is the component of \vec{E}_{UB} on y coordinate axis, and \vec{i}ₓ and \vec{i}_{y} are the unit vectors of the right-angled coordinate axis.

After the vector relationship of electric field vector is obtained, the equation (10) is calculated according to the equation (5). The following relationship equation is obtained.

$$( \frac{\partial {E}_{xB}}{\partial z} - \frac{\partial {E}_{zB}}{\partial x} ) {\vec{i}}_{y} = - \frac{ⅆ}{ⅆ t} B {\vec{i}}_{y} \qquad ( 11 )$$

where ∂E_{xB}/∂z is taking z as the partial derivative of component of \vec{E}_{UB} on x axis, ∂E_{zB}/∂y is taking x as the partial derivative of component of \vec{E}_{UB} on z axis, d/dt B is taking the differential of time by the magnetic flux when the electric field is changed.

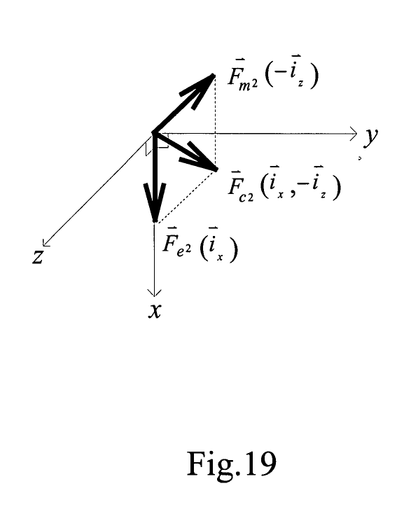

According to the equation (11), a magnetic field \vec{B} is induced by the time-varied electric field. When the pharmaceutical compound is positioned in the electric filed and the magnetic field inducted by the electric field, the pharmaceutical compound produces a force \vec{F}ₑ₂ of electric field due to the electric field of upper electrode 54, thereby having the pharmaceutical compound with a velocity v downwardly moving. When the force \vec{F}ₑ₂ affects the moving pharmaceutical compound, a force \vec{F}ₘ₂ is produced in the magnetic field induced by the electric field. The magnitude and direction of this force \vec{F}ₘ₂ of magnetic field are determined by the equation (3). According to the above-mentioned analysis, it found that the pharmaceutical compound is subjected to the force \vec{F}ₑ₂ of electric field and the force \vec{F}ₘ₂ of magnetic field, represented by the equation (4), a force \vec{F}_{c2} is interacted with these two forces. The magnitude and direction of this force \vec{F}_{c2} are the magnitude and direction of the vectors of \vec{F}ₑ₂ and \vec{F}ₘ₂ plus each other, as shown in FIG. 19, which is shown that the resulting force \vec{F}_{c2} is moving toward the skin. Thus, according to the above-mentioned analysis, a force is produced by the electric field profiled on the xy coordinate plane, thereby having the pharmaceutical compound with the kinetic energy moving toward the skin.

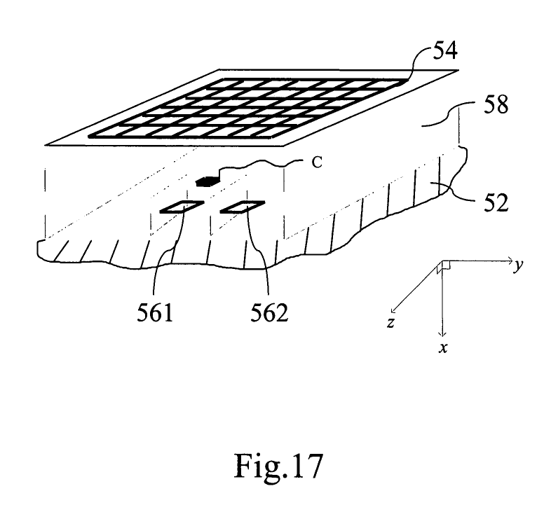

(c) The force which is applied on the pharmaceutical compound by the component of electric field having xy, yz and, zx coordinate planes, as shown in FIG. 17, when a pharmaceutical compound C is positioned on the xyz coordinate plane of lower electrode 561 and 562, a component of electric field is produced on this coordinate plane, and this component of electric field comprises three components x, y and z. The component of electric field caused by the lower electrode 562 from the upper electrode 54 is represented by the following equation (12).
\vec{E}_{UB}=E_{xB}(x,y,t)\vec{i}ₓ+E_{yB}(x,y,t)\vec{i}_{y}+E_{zB}(x,y,t)\vec{i}_{z} (12)
where \vec{E}_{UB} is the vector of electric field profile caused by the upper electrode 54 to the lower electrode 562, E_{xB} is the component of \vec{E}_{UB} on x coordinate axis, E_{yB} is the component of \vec{E}_{UB} on y coordinate axis, E_{zB} is the component of \vec{E}_{UB} on z coordinate axis and, \vec{i}ₓ, \vec{i}_{y} and \vec{i}_{z} are the unit vectors of the right-angled coordinate axis.

After the vector relationship of electric field vector is obtained, the equation (12) is calculated according to the equation (5). The following relationship equation is obtained.

$$( \frac{\partial {E}_{zB}}{\partial y} - \frac{\partial {E}_{yB}}{\partial z} ) {\vec{i}}_{x} + ( \frac{\partial {E}_{xB}}{\partial z} - \frac{\partial {E}_{zB}}{\partial x} ) {\vec{i}}_{y} + ( \frac{\partial {E}_{yB}}{\partial x} - \frac{\partial {E}_{xB}}{\partial z} ) {\vec{i}}_{z} = - \frac{ⅆ}{ⅆ t} B {\vec{i}}_{x} - \frac{ⅆ}{ⅆ t} B {\vec{i}}_{y} - \frac{ⅆ}{ⅆ t} B {\vec{i}}_{z} \qquad ( 13 )$$

Where ∂E_{zB}/∂y is taking y as the partial derivative of component of \vec{E}_{UB} on z axis, ∂E_{yB}/∂z is taking z as the partial derivative of component of \vec{E}_{UB} on y axis, ∂E_{xB}/∂z is taking z as the partial derivative of component of \vec{E}_{UB} on x axis, ∂E_{zB}/∂y is taking x as the partial derivative of component of \vec{E}_{UB} on z axis, ∂E_{xB}/∂y is taking x as the partial derivative of component of \vec{E}_{UB} on y axis, ∂E_{xB}/∂y is taking y as the partial derivative of component of \vec{E}_{UB} on x axis, d/dt B is taking the differential of time by the magnetic flux when the electric field is changed.

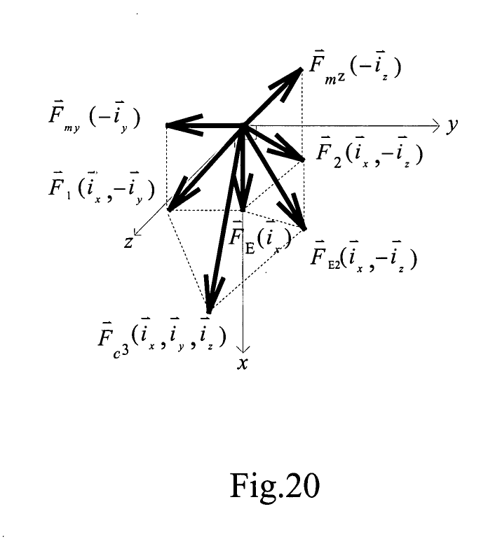

According to the equation (13), a magnetic field \vec{B} is induced by the time-varied electric field. When the pharmaceutical compound is positioned in the electric filed and the magnetic field inducted by the electric field, the pharmaceutical compound produces a force \vec{F}_{E} of electric field due to the electric field of upper electrode 54, thereby having the pharmaceutical compound with a velocity v downwardly moving. Thus, there are the components of x, y, and z coordinate in the magnitude field according to the equation (15). The forces of magnetic field comprise three forces of components \vec{F}ₘₓ, \vec{F}_{my}, and \vec{F}_{mz}, respectively. These three forces of components are interacted with the force \vec{F}_{E} of electric field. When interacting with the force \vec{F}_{E}, the force \vec{F}ₘₓ is parallel with the moving direction v (on the x coordinate axis) due to the direction of magnetic field (on the x coordinate axis), represented by the equation (3). The force \vec{F}ₘₓ will not be produced. Therefore, the pharmaceutical compound is only subjected to the force \vec{F}_{E}. Two forces \vec{F}_{my} and \vec{F}_{mz} are perpendicular to the force \vec{F}_{E}, thereby having the pharmaceutical compound with a force to move toward the skin. This force is conduced as same as the component of electric field of the above-mentioned charged particle on xy and xz coordinate. The forces profiled on xyz coordinate axis to the pharmaceutical compound are shown in the total resulting force \vec{F}_{C3} of the vector diagram of FIG. 20. In the vector diagram, if \vec{F}_{my}(−\vec{i}_{y}) is equal to \vec{F}_{mz}(−\vec{i}_{z}), these two forces are interacted with the force \vec{F}_{E}(\vec{i}ₓ) to produce the force \vec{F}₁(\vec{i}ₓ, −\vec{i}_{y}) and the force \vec{F}₂(\vec{i}ₓ, −\vec{i}_{z}), respectively. In order to combine the force \vec{F}₁(\vec{i}ₓ,−\vec{i}_{y}), \vec{F}₂(\vec{i}ₓ,−\vec{i}_{z}), and \vec{F}_{E}(\vec{i}ₓ) to a resulting force, the force \vec{F}₂(\vec{i}ₓ,−\vec{i}_{z}) and \vec{F}_{E}(\vec{i}ₓ) are combined to a resulting force \vec{F}_{E2}(\vec{i}ₓ,−\vec{i}_{z}), firstly. The force \vec{F}_{E2}(\vec{i}ₓ, −\vec{i}_{z}) and \vec{F}₁(\vec{i}ₓ,−\vec{i}_{y}) are combined to a total resulting force \vec{F}_{C3}. According to the vector diagram, the component direction of total resulting force \vec{F}_{C3} is toward the direction of the surface of skin. Therefore, the total resulting force \vec{F}_{C3} also causes the pharmaceutical compound to move toward the surface of skin.

According to the above-mentioned analysis, in the state 1, two-electrode structure comprises a force applied on the pharmaceutical compound, thereby having the pharmaceutical compound with a kinetic energy to move toward the direction of the skin.

(1) State 2 (t=t₁˜t₂)

In this state, 0 voltage is respectively applied on the upper electrode 54 and the lower electrode 561 and 562. There is no force applied on the pharmaceutical compound.

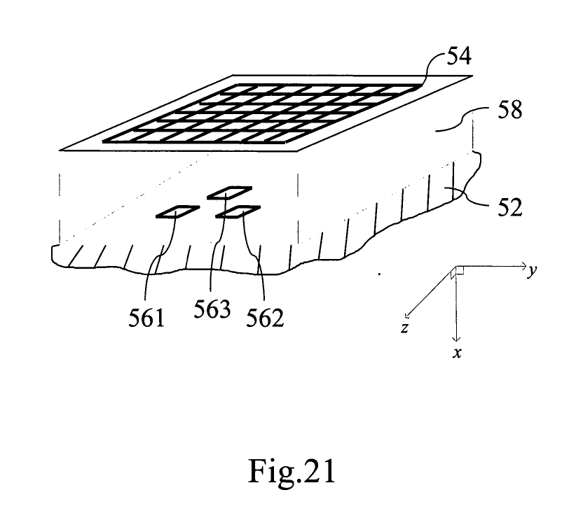

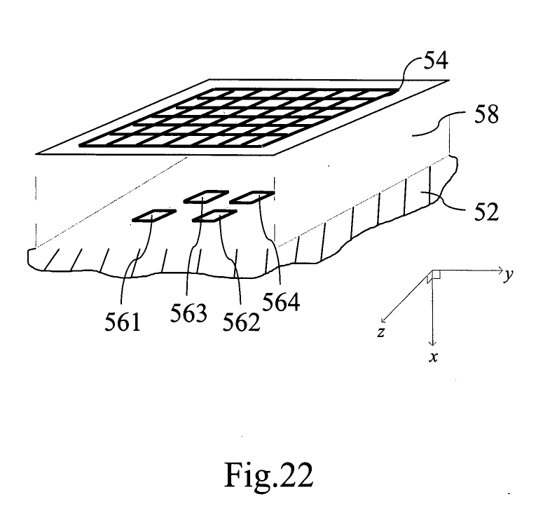

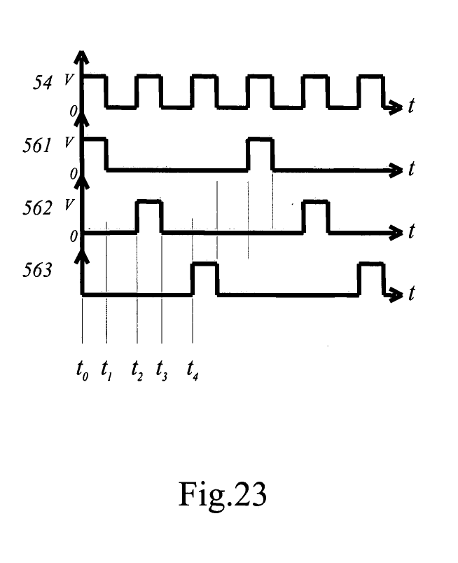

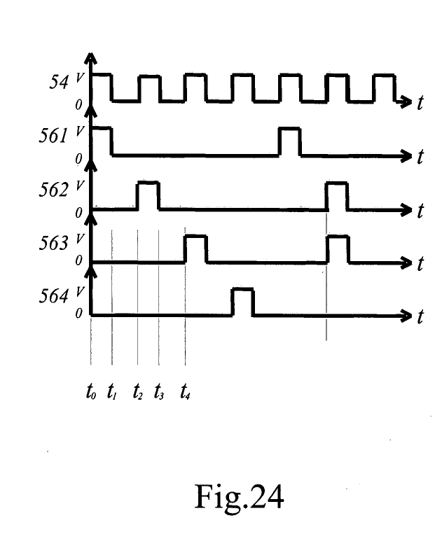

As a result, an alternating voltage is applied by using two electrodes to obtain a force toward the direction of skin. The same effect is obtained while changing the number of upper electrode and lower electrode. Even, the treatment area of the entire patch can be increased. Or, the voltage applied on the electrode can be reduced on the same treatment area. Even, the treatment area of the entire patch can be increased. Or, the voltage applied on the electrode can be reduced on the same treatment area in order to obtain the same effect between the electrodes while using the fewer electrodes. The multi-electrode structure, which is shown in FIG. 21, comprises a single set of upper electrode (the electromagnetic field electrode) 54 and three sets of lower electrodes (electroporation electrode) 561, 562, and 563. The multi-electrode structure, which is shown in FIG. 22, comprises a single set of upper electrode (the electromagnetic field electrode) 54 and four sets of lower electrodes (electroporation electrode) 561, 562, 563 and 564, and also uses the drive timing diagram shown in FIGS. 23 and 24 in order to drive three sets of electroporation electrodes and four sets of electroporation electrodes, respectively. The combination way of n sets of electrodes will be conducted depending on the combination of electrodes.

(II) The Drug with Negative Polarity

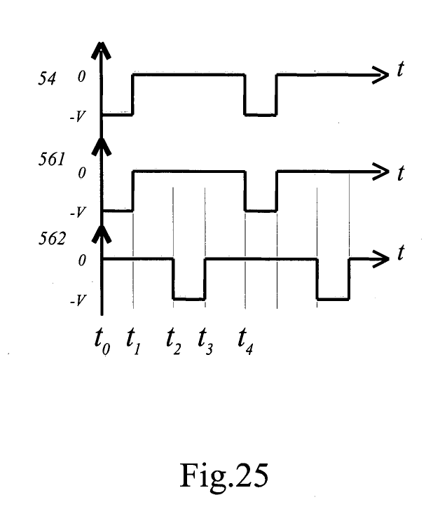

When the polarity of pharmaceutical compound which is going to deliver the human body is negative, the alternating electromagnetic field is produced if the drug is positioned between the upper electrode and the lower electrode and the time-varied voltage is applied on these two electrodes, so that the pharmaceutical compound has the kinetic energy moving toward the skin. For the lower electrode, due to the rotational electric field applied by the alternating voltage, an ion channel is formed in the surface of skin, so that the pharmaceutical compound is delivered into the human body more easily. In order to simplify the moving principle of the present invention herein, only one set of electrode is positioned in the upper electrode, and two sets of electrodes are positioned in the lower electrode. This combination is described by the architecture of electrodes of FIG. 13 and the drive timing of FIG. 25. As shown in FIG. 25, each drive period has four states. There are the differences of two states t₀˜t₁ and t₂˜t₃ in four states. There is no voltage applied on the lower electrode 561 and 562. The polarities of other electrodes are not changed. The drive timings t₀˜t₁ and t₂˜t₃ are moving the same way. The lower electrode 561 in the electric field is changed into the lower electrode 562, depending on the different state.

(1) State 1 (t=t₀˜t₁)

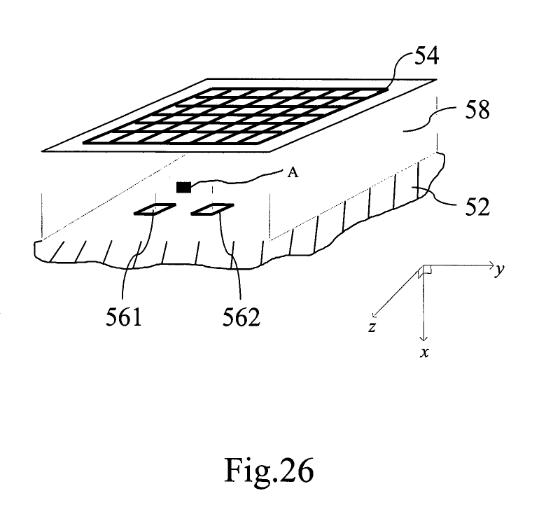

In this state, for the upper electrode 54 and the lower electrode 561, the negative voltages (−V) are applied on their voltage levels, and the voltage level and the timing-varied are the same. The variations in the electric field and the magnetic field are not changed between these tow electrodes. For the lower electrode 562, the applied voltage is zero level of voltage. There is a variation of voltage between the lower electrode 562 and the upper electrode 54, and there is a variation of alternating electric field. This variation of alternating electric field is changed between these two electrodes, depending on the plane position of the electrodes 561 and 562. The plane position of the pharmaceutical compound on the lower electrode 561 and 562 is changed as three different areas, respectively, as shown in FIGS. 26, 27 and 28. In these Figures, it can be seen that the vector of electric field is represented by the right-angled coordinate axis. The three different areas having the electric field profiles are analyzed as followings:

(a) The force which is applied on the pharmaceutical compound by the component of electric field having xy coordinate plane, as shown in FIG. 26, when a pharmaceutical compound A is positioned on the xy coordinate plane of lower electrode 561 and 562, a component of electric field is produced on this coordinate plane, and this component of electric field is profiled on the xy coordinate plane. The component of electric field caused by the lower electrode 562 from the upper electrode 54 is represented by the following equation (14).
\vec{E}_{BU}=E_{xB}(x,y,t)\vec{i}ₓ+E_{yB}(x,y,t)\vec{i}_{y} (14)
were \vec{E}_{BU} is the vector of electric field profile caused by the upper electrode 54 to the lower electrode 562, E_{xB} is the component of \vec{E}_{BU} on x coordinate axis, E_{yB} is the component of \vec{E}_{BU} on y coordinate axis, and \vec{i}ₓ and \vec{i}_{y} are the unit vectors of the right-angled coordinate axis.

After the relationship of electric field vector is obtained, the equation (14) is calculated according to the equation (15). The following relationship equation is obtained.

$$( \frac{\partial {E}_{yB}}{\partial x} - \frac{\partial {E}_{xB}}{\partial y} ) {\vec{i}}_{z} = - \frac{ⅆ}{ⅆ t} B {\vec{i}}_{z} \qquad ( 15 )$$

where ∂E_{yB}/∂x is taking x as the partial derivative of component of \vec{E}_{UB} on y axis, ∂E_{xB}/∂y is taking y as the partial derivative of component of \vec{E}_{UB} on x axis, d/dt B is taking the differential of time by the magnetic flux when the electric field is changed.

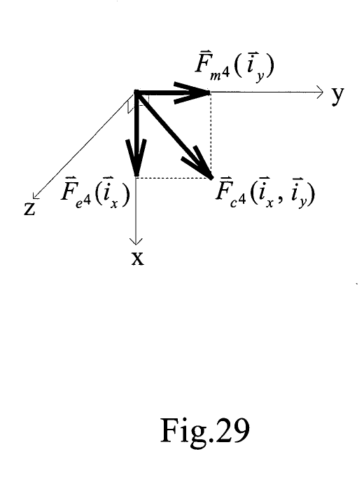

According to the equation (15), a magnetic field \vec{B} is induced by the time-varied electric field. When the pharmaceutical compound is positioned in the electric filed and the magnetic field induced by the electric field, the pharmaceutical compound produces a force \vec{F}ₑ₄ of electric field due to the electric field of upper electrode 54, thereby having the pharmaceutical compound with a velocity v downwardly moving. When the force \vec{F}ₑ₄ affects the moving pharmaceutical compound, a force \vec{F}ₘ₂ is produced in the magnetic field induced by the electric field. The magnitude and direction of this force \vec{F}ₘ₂ of magnetic field are determined by the equation (3). According to the above-mentioned analysis, it found that the pharmaceutical compound is subjected to the force \vec{F}ₑ₃ of electric field and the force \vec{F}ₘ₄ of magnetic field, represented by the equation (4), a force \vec{F}_{c4} is interacted with these two forces. The magnitude and direction of this force \vec{F}_{c4} are the magnitude and direction of the vectors of \vec{F}ₑ₃ and \vec{F}m3 plus each other, as shown in FIG. 29. In FIG. 29, it is seen that the direction of the force \vec{F}_{c4} is pointed toward the directions of lower electrode 561 and 562. If the bottom of lower electrode 561 and 562 is positioned on the skin, a force \vec{F}_{c3} is produced. According to the above-mentioned analysis, a force is produced by the electric field profiled on the xy coordinate plane, thereby having the pharmaceutical compound with the kinetic energy moving towards the skin.

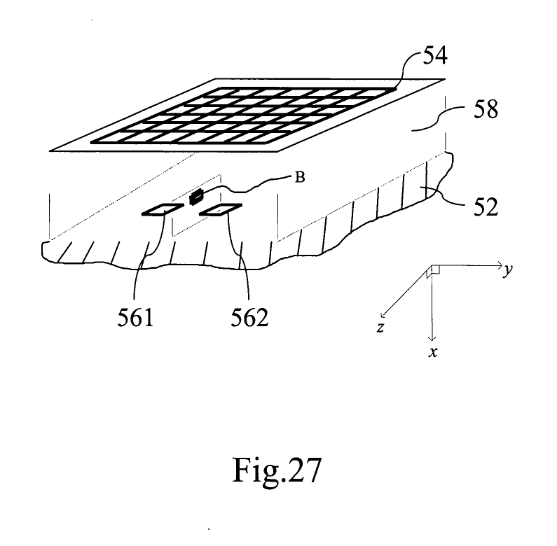

(b) The force which is applied on the pharmaceutical compound by the component of electric field having xz coordinate plane, as shown in FIG. 27, when a pharmaceutical compound B is positioned on the xz coordinate plane of lower electrode 561 and 562, a component of electric field is produced on this coordinate plane, and this component of electric field is profiled on the xz coordinate plane. The component of electric field caused by the lower electrode 562 from the upper electrode 54 is represented by the following equation (16).
\vec{E}_{BU}=E_{xB}(x,y,t)\vec{i}ₓ+E_{zB}(x,y,t)\vec{i}_{z} (16)
where \vec{E}_{BU} is the vector of electric field profile caused by the upper electrode 54 to the lower electrode 562, E_{xB} is the component of \vec{E}_{BU} on x coordinate axis, E_{zB} is the component of \vec{E}_{BU} on z coordinate axis, and \vec{i}ₓ and \vec{i}_{y} are the unit vectors of the right-angled coordinate axis.

After the vector relationship of electric field vector is obtained, the equation (16) is calculated according to the equation (5). The following relationship equation is obtained.

$$( \frac{\partial {E}_{xB}}{\partial z} - \frac{\partial {E}_{zB}}{\partial x} ) {\vec{i}}_{y} = - \frac{ⅆ}{ⅆ t} B {\vec{i}}_{y} \qquad ( 17 )$$

where ∂E_{xB}/∂z is taking z as the partial derivative of component of \vec{E}_{BU} on x axis, ∂E_{zB}/∂y is taking x as the partial derivative of component of \vec{E}_{BU} on z axis, d/dt \vec{B} is taking the differential of time by the magnetic flux when the electric field is changed.

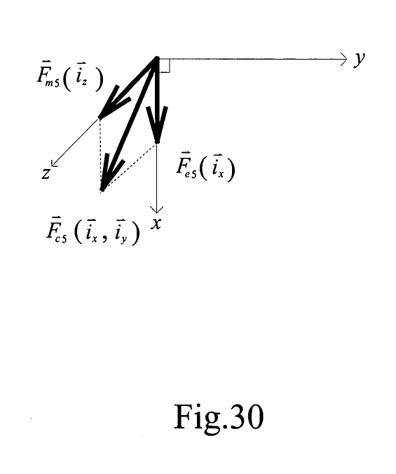

According to the equation (17), a magnetic fields \vec{B} is induced by the time-varied electric field. When the pharmaceutical compound is positioned in the electric filed and the magnetic field induced by the electric field, the pharmaceutical compound produces a force \vec{F}ₑ₅ of electric field due to the electric field of upper electrode 54, thereby having the pharmaceutical compound with a velocity v downwardly moving. When the force \vec{F}ₑ₅ affects the moving pharmaceutical compound, a force \vec{F}ₘ₅ is produced in the magnetic field induced by the electric field. The magnitude and direction of this force \vec{F}ₘ₂ of magnetic field are determined by the equation (3). Therefore, when the pharmaceutical compound is subjected to the force \vec{F}ₑ₅ of electric field and the force \vec{F}ₘ₅ of magnetic field, represented by the equation (4), a force \vec{F}_{c4} is interacted with these two forces. The magnitude and direction of this force \vec{F}_{c4} are the magnitude and direction of the vectors of \vec{F}ₑ₅ and \vec{F}ₘ₅ plus each other, as shown in FIG. 30, which is shown that the resulting force \vec{F}_{c4} is moving toward the skin.

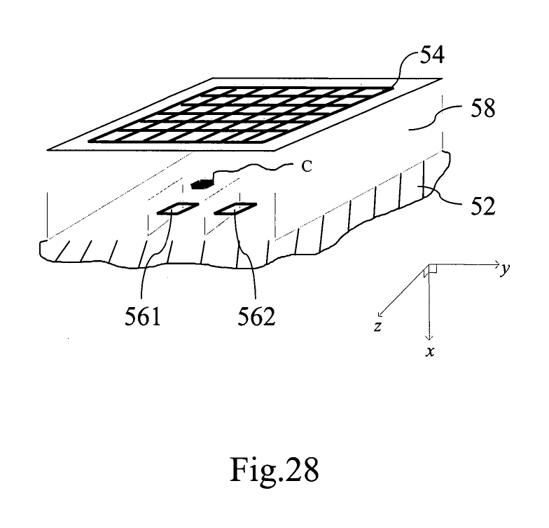

(c) The force which is applied on the pharmaceutical compound by the component of electric field having xy, yz and zx coordinate planes, as shown in FIG. 28, when a pharmaceutical compound C is positioned on the xyz coordinate plane of lower electrode 561 and 562, a component of electric field is produced on this coordinate plane, and this component of electric field comprises three components, x, y and z. The component of electric field caused by the lower electrode 562 from the upper electrode 54 is represented by the following equation (18).
\vec{E}_{BU}=E_{xB}(x,y,t)\vec{i}ₓ+E_{yB}(x,y,t)\vec{i}_{y}+E_{zB}(x,y,t)\vec{i}_{z} (18)
where \vec{E}_{BU} is the vector of electric field profile caused by the upper electrode 54 to the lower electrode 562, E_{xB} is the component of \vec{E}_{BU} on x coordinate axis, E_{yB} is the component of \vec{E}_{BU} on y coordinate axis, E_{zB} is the component of \vec{E}_{BU} on z coordinate axis, and ₓ, \vec{i}_{y} and \vec{i}_{z} are the unit vectors of the right-angled coordinate axis.

After the vector relationship of electric field vector is obtained, the equation (18) is calculated according to the equation (5). The following relationship equation is obtained.

$$( \frac{\partial {E}_{zB}}{\partial y} - \frac{\partial {E}_{yB}}{\partial z} ) {\vec{i}}_{x} + ( \frac{\partial {E}_{xB}}{\partial z} - \frac{\partial {E}_{zB}}{\partial x} ) {\vec{i}}_{y} + ( \frac{\partial {E}_{yB}}{\partial x} - \frac{\partial {E}_{xB}}{\partial z} ) {\vec{i}}_{z} = - \frac{ⅆ}{ⅆ t} B {\vec{i}}_{x} - \frac{ⅆ}{ⅆ t} B {\vec{i}}_{y} - \frac{ⅆ}{ⅆ t} B {\vec{i}}_{z} \qquad ( 19 )$$

where ∂E_{zB}/∂y is taking y as the partial derivative of component of \vec{E}_{BU} on z axis, ∂E_{yB}/∂z is taking z as the partial derivative of component of \vec{E}_{BU} on y axis, ∂E_{yB}/∂z is taking z as the partial derivative of component of \vec{E}_{BU} on x axis, ∂E_{zB}/∂y is taking y as the partial derivative of component of \vec{E}_{BU} on z axis, ∂E_{yB}/∂x is taking x as the partial derivative of component of \vec{E}_{UB} on y axis, ∂E_{xB}/∂y is taking y as the partial derivative of component of \vec{E}_{UB} on x axis, and d/dt B is taking the differential of time by the magnetic flux when the electric field is changed.

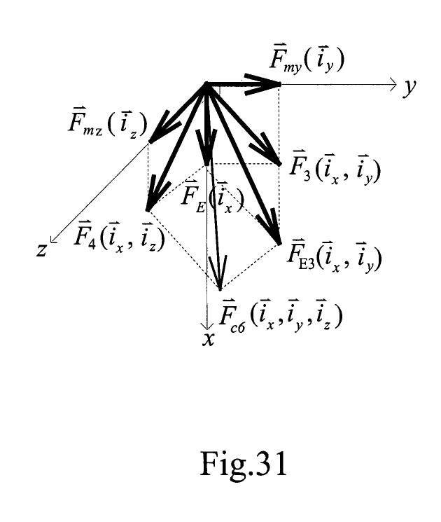

According to the equation (19), a magnetic field \vec{B} is induced by the time-varied electric field. When the pharmaceutical compound is positioned in the electric filed and the magnetic field inducted by the electric field, the pharmaceutical compound produces a force \vec{F}_{E} of electric field due to the electric field of upper electrode 54, thereby having the pharmaceutical compound with a velocity v downwardly moving. Thus, there are the components of x, y and z coordinate in the magnitude field according to the equation (19). The forces of magnetic field comprise three forces of components \vec{F}ₘₓ, \vec{F}_{my} and \vec{F}_{mz}, respectively. These three forces of components are interacted with the force \vec{F}_{E} of electric field. When interacting with the force \vec{F}_{E}, the force \vec{F}ₘₓ is parallel with the moving direction v (on the x coordinate axis) due to the direction of magnetic field (on the x coordinate axis), represented by the equation (3). The force \vec{F}ₘₓ will not be produced. Therefore, the pharmaceutical compound is only subjected to the force \vec{F}_{E}. Two forces \vec{F}_{my} and \vec{F}_{mz} are perpendicular to the force \vec{F}_{E}, thereby having pharmaceutical compound with a force to move toward the skin. This force is conduced as same as the component of electric field of the above-mentioned charged particle on xy and xz coordinate. The forces profiled on xyz coordinate axis to the pharmaceutical compound are shown in the total resulting force \vec{F}_{C6} of the vector diagram of FIG. 31. In the vector diagram, if \vec{F}_{my}(−\vec{i}_{y}) is equal to \vec{F}_{mz}(−\vec{i}_{y}), these two forces are interacted with the force \vec{F}_{E}(\vec{i}ₓ) to produce the force \vec{F}₃(\vec{i}ₓ, −\vec{i}_{y}) and the force \vec{F}₄(\vec{i}ₓ, −\vec{i}_{z}), respectively. In order to obtain a resulting force \vec{F}_{C6}, the force \vec{F}₃(\vec{i}ₓ,−\vec{i}_{y}) and \vec{F}_{E}(\vec{i}ₓ) are combined to a resulting force \vec{F}_{E3}(\vec{i}ₓ,\vec{i}_{y}), firstly. The force \vec{F}_{E3}(\vec{i}ₓ,\vec{i}_{y}) and \vec{F}₄(\vec{i}ₓ,\vec{i}_{y}) are combined to a total resulting force \vec{F}_{C6}. According to the vector diagram, the component direction of total resulting force \vec{F}_{C6} is toward the direction of the surface of skin. Therefore, the total resulting force \vec{F}_{C6} also causes the pharmaceutical compound to move toward the surface of skin.

According to the above-mentioned analysis, in the state 1, two-electrode structure comprises a force applied on the pharmaceutical compound, thereby having the pharmaceutical compound with a kinetic energy to move toward the direction of the skin.

(2) State 2 (t=t₁˜t₂)

In this state, 0 voltage is respectively applied on the upper electrode 54 and the lower electrode 561 and 562. There is no force applied on the pharmaceutical compound. As a result, the drugs with positive polarity and negative polarity or neutrality have the same function and effect. The various combination ways are used, and the details are omitted herein. The present invention provides an electric energy auxiliary device of transdermal drug delivery patch with a multi-electrode structure having drug delivery efficiency of iontophoresis, electroporation, and natural diffusion in order to provide the strongly thrust by using two-electrode structure to deliver the pharmaceutical compounds into the human body readily, not being subject to the size and hydrophile of the pharmaceutical compounds so as to improve effectiveness of drug delivery. While the invention has been particularly shown and described with reference to the preferred embodiments thereof, it will be understood by those skilled in the art that various changes in form and details may be made without departing from the spirit and scope of the invention.

What is claimed is:

1. An electric energy auxiliary device of transdermal drug delivery patch, which is attachable to the skin of a subject, comprising:
an upper electrode and a lower electrode or an upper electrode;
a drug storage area placed between said upper electrode and said lower electrode, thereby generating a kinetic energy in a pharmaceutical compound in said drug storage area toward a surface of the skin by a electric field between said upper electrode and said lower electrode, and said lower electrode generating an ion channel having the function of electroporation on said surface of the skin, or generating an ion channel the function of electroporation on the top of said lower electrode by said lower electrode on said surface of the skin;
at least one thin film positioned between said drug storage area and said lower electrode to control the release rate of said pharmaceutical compound; and
a adhesion layer positioned between said lower electrode and said thin film to cover the top surface of said lower electrode, thereby being attachable to the skin to contact with the bottom of said lower electrode.

2. The electric energy auxiliary device of transdermal drug delivery patch according to claim 1, comprising a layer of a backing covered over the outer surface of said upper electrode.

3. The electric energy auxiliary device of transdermal drug delivery patch according to claim 1, wherein said upper electrode and said lower electrode are selected from the group consisting of single set of electrode and a plurality sets of electrodes.

4. The electric energy auxiliary device of transdermal drug delivery patch according to claim 1, wherein the voltage applied on said upper electrode and said lower electrode is a time-varied voltage.

5. The electric energy auxiliary device of transdermal drug delivery patch according to claim 1, wherein aid upper electrode and said lower electrode are respectively formed of any shape of electrode array.

6. An electric energy auxiliary device of transdermal drug delivery patch, which is attachable to the skin of a subject, comprising:
an upper electrode and a lower electrode; and
a drug storage area placed between said upper electrode and said lower electrode, thereby generating a kinetic energy in a pharmaceutical compound in said drug storage area toward a surface of the skin by a electric field between said upper electrode and said lower electrode, and said lower electrode generating a ion channel having the function of electroporation on said surface of the skin.

7. The electric energy auxiliary device of transdermal drug delivery patch according to claim 6, wherein at least one thin film is positioned between said drug storage area and said lower electrode to control the release rate of said pharmaceutical compound.

8. The electric energy auxiliary device of transdermal drug delivery patch according to claim 6, wherein a adhesion layer is positioned between said lower electrode and said thin film to cover the top surface of said lower electrode, thereby being attachable to the skin to contact with the bottom of said lower electrode.

9. The electric energy auxiliary device of transdermal drug delivery patch according to claim 6, wherein a layer of a backing is covered over the outer surface of said upper electrode.

10. The electric energy auxiliary device of transdermal drug delivery patch according to claim 6, wherein said upper electrode and said lower electrode are selected from the group consisting of single set of electrode and a plurality sets of electrodes.

11. The electric energy auxiliary device of transdermal drug delivery patch according to claim 6, wherein the voltage applied on said upper electrode and said lower electrode is a time-varied voltage.

12. The electric energy auxiliary device of transdermal drug delivery patch according to claim 1, wherein aid upper electrode and said lower electrode are respectively formed of any shape of electrode array.
